(19) United States
(12) Patent Application Publication
(10) Pub. No.: US 2024/0312624 A1
(43) Pub. Date: Sep. 19, 2024

(54) Method for Updating Infusion Control Parameter, Device, Infusion Pump and Storage Medium

(71) Applicant: Medcaptain Medical Technology Co., Ltd., Shenzhen (CN)

(72) Inventors: Shengping LI, Shenzhen (CN); Ping LI, Shenzhen (CN); Longyu WU, Shenzhen (CN); Yazhou TANG, Shenzhen (CN)

(73) Assignee: Medcaptain Medical Technology Co., Ltd., Shenzhen (CN)

(21) Appl. No.: 18/605,811

(22) Filed: Mar. 14, 2024

(30) Foreign Application Priority Data
Mar. 15, 2023 (CN) 202310284903.6

(51) Int. Cl. G16H 40/67; A61M 5/142; A61M 5/172; G16H 20/17

(52) U.S. Cl. CPC G16H 40/67; A61M 5/142; A61M 5/1723; G16H 20/17

(57) ABSTRACT

The invention provides a method for updating an infusion control parameter, a device, an infusion pump and a storage medium, and relates to the technical field of medical devices. The infusion pump may analyze the obtained infusion record and/or medical advice information to obtain an alternative control parameter corresponding to at least one drug name. Finally, the infusion pump can update a drug database stored in the infusion pump according to the alternative control parameters corresponding to the obtained at least one drug name. There is no need for operation and maintenance staff to collect drug information and infusion control parameters for infusion from doctors, pharmacists, nurses and other medical staff, thereby saving a lot of labor cost and time cost.

### REFERENCE TO RELATED APPLICATIONS

This application claims priority of Chinese Patent Application No. 202310284903.6, entitled “Method for Updating Infusion Control Parameter, Device, Infusion Pump and Storage Medium” filed with the China National Intellectual Property Administration on Mar. 15, 2023, which is incorporated herein by reference in its entirety.

### TECHNICAL FIELD

The invention relates to the technical field of medical devices, and more particularly, to a method for updating an infusion control parameter, device, infusion pump and storage medium.

### BACKGROUND ART

An infusion pump is a kind of intelligent infusion device, in particular, specialized medical equipment that uses a mechanical drive to accurately control the infusion rate of an infusion tube to ensure that an output infusion dose is accurate and safe.

Currently, when tracing back the history of medication administration by medical staff, it is necessary for the medical staff to manually enter information and usage statistics relating to a drug, including the drug's name, the drug concentration, the infusion rate, the infusion volume, etc. This incurs a large cost in terms of labor and time for medical staff such as doctors, pharmacists or nurses.

### SUMMARY OF THE INVENTION

The invention provides a method for updating an infusion control parameter, a device, an infusion pump, and a storage medium for solving the problems of high labor and time costs for updating a drug database of an infusion pump, and low content richness and poor reliability of the drug database in the prior art.

In a first aspect, the invention provides a method for updating an infusion control parameter and applying it to control the operation of an infusion pump. The method provided by this invention comprises:

obtaining an infusion record and/or information relating to medical advice in an infusion pump satisfying at least one selected analysis rule, wherein the infusion record comprises at least one drug name and a corresponding infusion record; the infusion record further comprises at least one infusion control parameter, and the medical advice information comprises at least one drug name and a corresponding infusion control parameter;
analyzing the obtained infusion record and/or medical advice information to obtain an alternative control parameter corresponding to at least one drug name; and
updating a drug database stored in the infusion pump according to the obtained alternative control parameters corresponding to the at least one drug name.

In a possible embodiment, the step of analyzing the obtained infusion record and/or medical advice information to obtain the alternative control parameter corresponding to at least one drug name comprises:
analyzing a change and/or frequency of use of the at least one infusion control parameter corresponding to at least one the drug name in the obtained infusion record and/or medical advice information, to obtain the alternative control parameter corresponding to the at least one drug name;
or, analyzing a difference between the at least one infusion control parameter in the infusion record corresponding to at least one drug name, and the infusion control parameter corresponding to the drug name in the medical advice information and/or the infusion control parameter corresponding to the drug name stored in the infusion pump to obtain the alternative control parameter corresponding to the at least one drug name;
or, analyzing a difference between the infusion control parameter in the medical advice information corresponding to the at least one drug name and the infusion control parameter corresponding to the drug name stored in the infusion pump to obtain the alternative control parameter corresponding to the at least one drug name.

In a possible embodiment, the infusion record comprises patient information and/or infusion consumable information; the alternative control parameter is associated with one or more of a patient type, a disease type, a medical history, a treatment history, departmental information, a consumable type, a consumable model, and a consumable specification.

In a possible implementation, the selected analysis rule comprises one or more of an infusion time interval, an infused drug name, department information, a patient type, a disease type, a number of infusions, a frequency of changes to the infusion control parameter, a consumable type, a consumable model, a consumable specification, and a user trigger.

In a possible embodiment, after the step of analyzing the obtained infusion records and/or medical advice information to obtain the alternative control parameters corresponding to the at least one drug name, the method further comprises:
outputting the obtained at least one drug name and its corresponding infusion control parameter.

In a possible embodiment, the method provided by the invention further comprises:
outputting an alarm message when detecting that the frequency of use of the alternative control parameter corresponding to the at least one drug name in the drug database is lower than a set frequency threshold.

In a possible embodiment, after the step of analyzing the obtained the infusion record and/or medical information to obtain the alternative control parameter corresponding to the at least one drug name, the method further comprises:
outputting the obtained at least one drug name and its corresponding alternative control parameter to one or more of an input-output device, other infusion pumps, a workstation, and a central station.

In a second aspect, the invention further provides a device for updating the infusion control parameter comprising:
a data acquisition unit configured to obtain an infusion record collection and/or medical advice information in an infusion pump satisfying at least one selected analysis rule, wherein the infusion record collection comprises an infusion record that corresponds to at least one drug name; the infusion record further comprises at least one infusion control parameter, and the medical advice information comprises at least one drug name and a corresponding infusion control parameter

a data analysis unit configured to analyze the obtained infusion record and/or medical advice information to obtain an alternative control parameter corresponding to at least one drug name; and

a data updating unit configured to update a drug database stored in the infusion pump according to the obtained alternative control parameters corresponding to the at least one drug name.

In a third aspect, the invention further provides an infusion pump comprising a driving device, a casing, and a storage device arranged in the casing, a controller configured to control the driving device to push out a liquid in an infusion consumable connected to the infusion pump, wherein the controller performs a method, comprising:
obtaining an infusion record collection and/or medical advice information in an infusion pump satisfying at least one selected analysis rule, wherein the infusion record collection comprises an infusion record that corresponds to at least one drug name; the infusion record further comprises at least one infusion control parameter, and the medical advice information comprises at least one drug name and a corresponding infusion control parameter;
analyzing the obtained infusion record and/or medical advice information to obtain an alternative control parameter corresponding to at least one drug name; and
updating a drug database stored in the infusion pump according to the obtained alternative control parameters corresponding to the at least one drug name.

In a fourth aspect, the invention further provides a computer-readable storage medium, the computer-readable storage medium storing a computer program, wherein the computer program, when executed by the controller, causes the computer to perform a method, comprising:
obtaining an infusion record collection and/or medical advice information in an infusion pump satisfying at least one selected analysis rule, wherein the infusion record collection comprises an infusion record that corresponds to at least one drug name; the infusion record further comprises at least one infusion control parameter, and the medical advice information comprises at least one drug name and a corresponding infusion control parameter;
analyzing the obtained infusion record and/or medical advice information to obtain an alternative control parameter corresponding to at least one drug name; and
updating a drug database stored in the infusion pump according to the obtained alternative control parameters corresponding to the at least one drug name.

The invention provides a method for updating an infusion control parameter, a device, an infusion pump, and a storage medium, such that the infusion pump can automatically obtain an infusion record and/or medical advice information satisfying at least one selected analysis rule. The obtained infusion record and/or medical advice information is analyzed to obtain an alternative control parameter corresponding to at least one drug name, and the drug database stored in the infusion pump can be updated according to the obtained alternative control parameter corresponding to the at least one drug name, thereby eliminating the need for the operation and maintenance personnel to collect drug information and infusion control parameters for the infusion from medical personnel such as doctors, pharmacists, nurses, etc., and saving a large amount of labor cost and time cost.

### BRIEF DESCRIPTION OF THE DRAWINGS

In order to more clearly illustrate the technical solutions in the embodiments of the invention or prior art, the accompanying drawings used in the description of the embodiments or prior art will be briefly introduced as follows. Obviously, the accompanying drawings in the following description depict only some of the embodiments of the invention, and a person of ordinary skill in the field would be able to envision even other embodiments without need for inventive effort.

FIG. 1 is a flowchart of a method for updating an infusion control parameter according to an embodiment of the invention.

FIG. 2 is a block diagram of a circuit connection of an infusion pump according to an embodiment of the invention.

FIG. 3 shows a flowchart of a device for updating the infusion control parameter according to an embodiment of the invention.

### DETAILED DESCRIPTION

In order to make the purpose, technical solutions and advantages of the embodiments of the invention clearer, these will be described clearly and completely in the following in conjunction with the accompanying drawings of embodiments of the invention. It is clear, however, that the described embodiments only some of those that are possible, and not all embodiments. Other embodiments made by a person of ordinary skill in the art, guided by the description of the embodiments herein, fall within the scope of protection of the invention.

The terms “first”, “second”, “third”, “fourth” and the like (if any) in the specification and the claims of the invention and the above-described drawings are used to distinguish similar objects and do not require to describe a particular order or sequence. It should be understood that the terms used may be interchangeable, where appropriate, so that the embodiments of the invention described herein can be implemented in an order other than those illustrated or described herein. In addition, the terms “comprising” and “having”, and any changes thereof, are intended to cover non-exclusive embodiments, e.g., processes, methods, systems, products or equipment including a series of steps or units need not be limited to those clearly listed, but may include other steps or units that are not clearly listed or that are inherent to those processes, methods, products or equipment.

In the method for updating infusion control parameters provided by the invention, the infusion pump can automatically obtain an infusion record and/or medical advice information, that is, orders and other information given by a doctor to a patient satisfying at least one selected analysis rule. The obtained infusion record and/or medical advice information is analyzed to obtain an alternative control parameter corresponding to at least one drug name, and the drug database stored in the infusion pump can be updated according to the obtained alternative control parameter corresponding to the at least one drug name, thereby eliminating the need for the operation and maintenance personnel, such as user service staff and the equipment manufacturer, to collect drug information and infusion control parameters for the infusion from medical personnel such as doctors, pharmacists, nurses, etc., and saving a large amount of labor cost and time cost.

In the following, the technical solutions of the invention and how they solve the above technical problems are described in detail by means of description of specific embodiments. The following specific embodiments may be combined with each other, and the same or similar concepts or processes may be omitted in some embodiments. Embodiments of the invention will be described below in conjunction with the accompanying drawings.

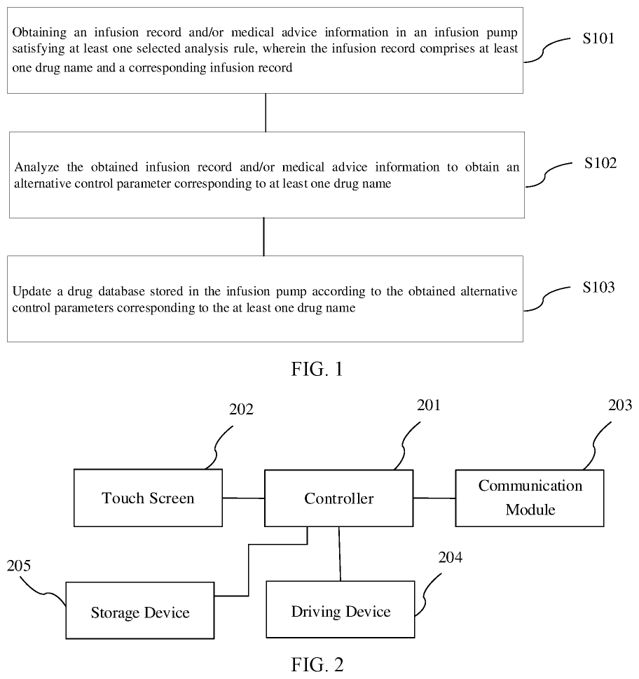

As shown in FIG. 1, embodiments of the invention provide a method for updating an infusion control parameter, and applying it to control the operation of an infusion pump. The infusion pump is an intelligent infusion device, a specialized medical device that uses mechanical driving force to accurately control the infusion rate of the output drug in the infusion tube to ensure that the output drug dose is accurate and safe. Specifically, as shown in FIG. 2, the infusion pump may include a casing, a controller 201, an input-output device 202, a communication module 203, a driving device 204, and a storage device 205. The controller 201, the communication module 203, the driving device 204, and the storage device 205 are arranged in the casing. The controller 201 is electrically connected to the input-output device 202, the communication module 203, and the driving device 204, and the storage device 205, respectively, and the controller 201 is configured to control the driving device 204 to output the medicinal fluid in the infusion consumable.

The input-output device 202 may be configured to input at least one drug name and a corresponding infusion record. As a non-limiting example, the input-output device 202 may include one or more of a touch screen, a keyboard, a keypad, and a scanning gun. The communication module 203 may be configured to receive infusion control parameters and/or medical advice information, etc., from a remote end system such as a server, a doctor's terminal, etc. The communication module 203 may be a wireless communication module and/or interface. In order to apply to different infusion drugs, scenarios, patients, etc., the infusion consumables have different types, such as syringes, infusion tubes, etc. The same type of infusion consumables may also have different specifications, sizes, models, etc. Specifically, as shown in FIG. 1, the method provided by an embodiment of the invention includes the following steps.

S101: an infusion record and/or medical advice information in the infusion pump satisfying at least one selected analysis rule is obtained, in which the infusion record includes at least one drug name and a corresponding infusion record.

It should be noted that the infusion record and/or medical advice information may, as non-limiting examples, be historical information stored in the infusion pump's storage device 205, may be input by healthcare personnel using the input-output device 202 of the infusion pump, or may be acquired remotely or scanned by a wireless communication module or interface.

Additionally, the selected analysis rules may include one or more selected conditions such as an infusion time interval, an infused drug name, department information, a patient type, a disease type, a number of infusions, a change frequency of infusion control parameter change, a user trigger, a consumable type, a consumable model, a consumable specification, and the like. The department information may include department information that may record the use of the infusion pump for the infusion operation. The department information may be the name of a section of a department, or the name of a large department that includes multiple departments. The patient type may refer to the basic classification information of the patient, which may include one or more of the patient's gender, the patient's age range, the ethnicity of the patient, and so on. For example, if the selected analysis rule is an infusion time interval (e.g., January 1-January 31), the infusion records and/or medical advice information in the infusion pump that satisfies the requirements in the infusion time interval (e.g., January 1-January 31) is obtained. As another example, if the selected analysis rule is the department information (e.g., internal medicine), the infusion records and/or medical advice information in the infusion pump that satisfy the corresponding department information (e.g., Internal Medicine) is input.

In this way, the user can filter for the infusion records and/or medical advice information to be analyzed for subsequent analysis by setting one or more selected conditions of the selected analysis rule according to personal or practical needs. The selected analysis rules may be stored in advance in the storage device 205 of the infusion pump, or they may be input by the user at the time of analysis or obtained remotely, which is not limited in the invention.

When the selected conditions for the selected analysis rules include an infusion time interval, the infusion records from infusion pumps where the time of execution of the infusion operation meets the corresponding infusion time interval are selected. The medical advice information stored in the infusion pump at a time that satisfies the corresponding infusion time interval is then selected. The time of the medical advice information may be the time when the medical advice information is generated or the time when the infusion pump receives the medical advice information, which may be set according to the practical requirement. When the selected conditions of the selected analysis rule include department information, it may be that the department in which the infusion pump performs the infusion operation is selected to satisfy the infusion record corresponding to the department information, or it may be that the department in which the infusion pump prescribes the infusion operation is selected to satisfy the infusion record corresponding to the department information. For example, it might be that at least one medication corresponding to the drug name is prescribed by the department corresponding to the department name in which the medication is administered. The medication information may include the department information that prescribed the corresponding medication information, a doctor, and other information.

The infusion records include at least one infusion control parameter, the infusion control parameter includes an infusion control parameter name and a corresponding value thereof, and the medical advice information includes the at least one drug name and the corresponding infusion control parameter.

As a non-limiting example, the infusion record may include a drug name A and a corresponding infusion record, a drug name B and a corresponding infusion record, and a drug name C and a corresponding infusion record, etc. . . . The medical advice information may include the drug name A and the corresponding infusion control parameter, the drug name B and the corresponding infusion control parameter, and the drug name C and the corresponding infusion control parameter.

The infusion control parameter may include one or more of a drug concentration parameter, an infusion rate parameter, an infusion volume parameter, and an infusion time parameter. For example, when the infusion control parameter includes the drug concentration parameter and the infusion rate parameter, the infusion pump control driving device outputs the drug solution in the infusion consumable to the patient in accordance with the drug concentration parameter and the infusion rate parameter.

Further, the drug concentration parameter may include one or more of a first drug concentration, a drug concentration range, a drug concentration threshold, and the like. The first drug concentration may be the drug concentration during use corresponding to the drug named in the infusion record, e.g., concentration L1. The drug concentration range may be a permissible drug concentration range corresponding to the drug named in the infusion record, such as (L2, L3). The drug concentration threshold is an upper limit value (e.g., L2) and/or a lower limit value (L3) of the permissible drug concentration corresponding to the drug named in the infusion records. The infusion rate parameter may include one or more of a first infusion rate, an infusion rate range, an infusion rate threshold, and the like. The first infusion rate may be an infusion rate during use corresponding to the drug named in the infusion record, e.g., flow rate V1. The infusion rate range may be a range of permissible infusion rates corresponding to the drug named in the infusion record, e.g., (V2, V3). The infusion rate threshold may be an upper limit value (e.g., V2) and/or a lower limit value (V3) of the permissible infusion rate corresponding to the drug named in the infusion records. The infusion volume parameter may include one or more of a first infusion volume, an infusion volume range, an infusion volume threshold, and the like. The infusion volume parameter is an infusion volume during use corresponding to the drug named in the infusion record, e.g., infusion volume N1. The infusion volume range is a range of permissible infusion volume corresponding to the drug named in the infusion records, e.g., (N2, N3). The infusion volume threshold is an upper limit value (e.g., N2) and/or a lower limit value (N3) of the permissible infusion volume corresponding to the drug named in the infusion records. The infusion duration includes one or more of: an infusion duration range, an infusion duration threshold, and an infusion period. The infusion duration range may be a range of permissible infusion durations corresponding to the drug named in the infusion records, e.g. (T2, T3). The infusion duration threshold is an upper limit (e.g., T2) and/or lower limit (T3) of the permissible infusion duration for the drug named in the infusion record. The infusion time period is the time period (e.g., 9:00-10:00) corresponding to the drug named in the infusion record at the time of use.

It should be noted that the above-described first drug concentration may be a preferably recommended drug concentration, and the drug concentration parameter may also include a second drug concentration, a third drug concentration, etc. The second drug concentration may be a secondarily recommended drug concentration, and the third drug concentration may be a third-choice recommendation for the drug concentration. Similarly, the above-described first infusion rate may be a preferably recommended infusion rate, and the infusion rate parameter may also include a second infusion rate, a third infusion rate, etc. The second infusion rate may be a secondarily recommended infusion rate, the third infusion rate may be third-choice recommendation for the infusion rate. The above-described first infusion duration may be a preferably recommended infusion duration, and the infusion duration parameter may also include a second infusion duration and a third infusion duration, etc. The second infusion duration may be a secondarily recommended infusion duration, and the third infusion duration may be third-choice recommendation for the infusion duration.

S102: The obtained infusion record and/or medical advice information is analyzed to obtain alternative control parameters corresponding to at least one drug name.

Specifically, the specific realization of S102 includes, but is not limited to, the following procedures.

The first procedure: the changes and/or the frequency of use of the values of the at least one infusion control parameter corresponding to the at least one drug name in the obtained infusion record and/or the medical advice information are analyzed, to obtain the alternative control parameter corresponding to the at least one drug name.

The step of analyzing the changes and/or frequency of use of the values of the at least one infusion control parameter corresponding to the at least one drug name in the obtained infusion record and/or the medical advice information includes: analyzing the changes and/or frequency of use of the values of the at least one infusion control parameter in at least one infusion record, which includes analyzing the values of the at least one infusion control parameter separately for different infusion records, or analyzing the values of the at least one infusion control parameter jointly for different infusion records. These changes in the infusion control parameters may be automatically adjusted by the infusion pump, or adjusted by the infusion pump in response to received control information.

The step of analyzing different infusion records separately includes separately analyzing the changes and/or the frequency of use in the values of the same infusion control parameter in at least one infusion record to obtain the alternative control parameter corresponding to at least one drug name, which specifically may include at least one infusion control parameter and its corresponding alternative value. For example, for a drug name, the value of the last-used infusion control parameter in an infusion record may be used as the alternative value of the infusion control parameter; the value of the infusion control parameter that has been used for the longest period of time or has been used with the highest frequency in an infusion record may be used as the alternative value of the infusion control parameter; or the analysis rules may be chosen according to the practical requirements, such as determine the alternative value of the at least one infusion control parameter in combination with weights, other infusion control parameters, control timing, etc.

The step of analyzing different infusion operations jointly includes analyzing the changes and/or the frequency of use in the values of the same infusion control parameter in the at least two infusion records to obtain the alternative control parameter corresponding to the at least one drug name. For example, for a drug name, the value of the last infusion control parameter used in the multiple infusion records may be used as the alternative value of the infusion control parameter; the value of the infusion control parameter with the highest frequency or the longest duration of use in the multiple infusion records may be used as the alternative value of the infusion control parameter; or the analysis rules may be set up according to the practical requirements, such as determine the at least one alternative control parameter corresponding to the at least one drug name in combination with weights, other infusion control parameters, control timing, etc.

The second procedure: a difference between the at least one infusion control parameter in the infusion record corresponding to the at least one drug name, and the infusion control parameter corresponding to the drug named in the medical advice information and/or the infusion control parameter corresponding to the drug name stored in the infusion pump is analyzed, in order to obtain the alternative control parameter corresponding to the at least one drug name.

One way to analyze at least one infusion control parameter in the infusion record corresponding to the at least one drug name to obtain a preselected control parameter corresponding to the at least one drug name, and a difference between the obtained preselected control parameter and the infusion control parameter corresponding to the drug name in the medical advice information and/or the infusion control parameter corresponding to the drug name stored in the infusion pump is by comparing them. If the difference is greater than a preset first difference threshold value, then the preselected control parameter is used as an alternative control parameter. In this circumstance, the method of analyzing the at least one infusion control parameter in the infusion record corresponding to the at least one drug name to obtain the pre-selected control parameter corresponding to the at least one drug name is as described above for obtaining the alternative control parameter, which will not be repeated.

Of course, it is also possible to analyze a difference between the changes of the at least one infusion control parameter in the infusion record corresponding to the at least one drug name and the infusion control parameter corresponding to the drug name in the medical advice information and/or the infusion control parameter corresponding to the drug name stored in the infusion pump. For example, if a difference between the at least one infusion control parameter and the infusion control parameter corresponding to the drug name in the medical advice information and/or the infusion control parameter corresponding to the drug name stored in the infusion pump becomes larger, the alternative control parameter is determined according to the infusion control parameter. If the difference becomes smaller, the infusion control parameter corresponding to the drug name in the medical advice information and/or the infusion control parameter corresponding to the drug name stored in the infusion pump is determined as the alternative control parameter. Of course, the above method may be adjusted according to practical requirements, which is not limited in the embodiments of the present application.

A third procedure: analyzing a difference between the infusion control parameter in the medical advice information corresponding to the at least one drug name and the infusion control parameter corresponding to the drug name stored in the infusion pump, to obtain the alternative control parameter corresponding to the at least one drug name.

The difference between the alternative control parameter or the infusion control parameter corresponding to the same drug name according to the change of the same drug name in the medical advice information and the infusion control parameter corresponding to the drug name stored in the infusion pump may be analyzed. For example, the difference between the infusion control parameter with the highest frequency of occurrence and the stored infusion control parameter is analyzed. If the difference exceeds a second difference threshold, then the infusion control parameter with the highest frequency of occurrence is used as the alternative control parameter. Alternatively, the difference between the alternative control parameter corresponding to the same drug name in the latest medical advice information and the stored infusion control parameter is analyzed. If the difference exceeds the second difference threshold, then the infusion control parameter with the highest frequency of occurrence is taken as the alternative control parameter.

It can be understood that one of the above rules may be optionally selected according to the needs of the user to obtain at least one alternative control parameter corresponding to the drug name, which is convenient and flexible.

In addition, the infusion record may also include patient information. The patient information may include one or more of a patient type, a disease type, a medical history, a treatment history, departmental information, and the like. For example, the patient type may include one or more of: a patient age group, gender, ethnicity, and the like. In this circumstance, the alternative control parameter corresponding to the at least one drug name obtained in S102 is associated with one or more of the patient type, the disease type, the treatment history, or the department information. The patient type, disease type, treatment history, or department information may be derived from patient information.

For example, the infusion record records that drug A was infused to patient A under infusion control parameter A; drug A was infused to patient B under infusion control parameter B; drug A was infused to patient C under infusion control parameter C; drug A was infused to patient D under infusion control parameter B, and so on. Then, by combining the patient information of different patients in the infusion record, the alternative control parameters of the drug A corresponding to one or more different combinations of patient type, disease type, treatment history and departmental information may be obtained. That is, the step of analyzing the infusion record to obtain the alternative control parameters of at least one drug obtained are associated with one or more different combinations of the patient type, disease type, treatment history and department information.

Of course, the medical advice information may also include patient information. Similarly, the step of analyzing the medical advice information or the infusion information and the medical advice information to obtain the alternative control parameter corresponding to the at least one drug name may also be associated with one or more of the patient type, the disease type, the medical history, the treatment history, the departmental information, and the consumable type, the consumable model, and the consumable specification.

In addition, the infusion record may also include infusion consumable information, such as one or more of: the consumable type, the consumable specification, the consumable model, and the like. In this circumstance, the at least one drug name obtained in S102 corresponding to the alternative control parameters are associated with one or more of the consumable type, consumable model, and consumable specification.

For example, assume that the infusion record records that drug A was infused to patient A using infusion consumable A under infusion control parameter A; drug A was infused to patient B using infusion consumable B under infusion control parameter B; drug A was infused to patient C using infusion consumable C under infusion control parameter C; and drug A was infused to patient D using infusion consumable D under infusion control parameter D. Then, by combining the infusion consumable information used in the infusion record, the alternative control parameters of the drug A may be obtained for different combinations of one or more of the corresponding consumable types, consumable specifications, consumable models, etc. That is, the alternative control parameter of the at least one drug obtained by analyzing the infusion record is associated with different combinations of one or more of the consumable types, consumable specifications, and consumable models.

Further, if the infusion record includes patient information and infusion consumable information, the alternative control parameter corresponding to the at least one drug name obtained in S102 is associated with one or more of the patient type, the disease type, the treatment history, the department information, the consumable type, the consumable model, and the consumable specification.

S103: the drug database stored in the infusion pump is updated according to the obtained alternative control parameter corresponding to the at least one drug name.

The updating may include adding and replacing.

For example, when a drug name is not included in the stored drug database, the drug name and its corresponding alternative control parameter may be added to the drug database.

For another example, when a certain drug name is included in the drug database stored by the infusion pump, but the alternative control parameter corresponding to the drug name analyzed in S102 is different from the infusion control parameter stored in the drug database, the infusion control parameter corresponding to the drug name in the drug database is replaced with the obtain alternative control parameter by analyzing.

As a non-limiting example, if a certain drug name may correspond to a variety of infusion control parameters in the drug database stored in the infusion pump, when the obtain alternative control parameter by analyzing corresponding to the drug name in S102 is different from the infusion control parameter stored in the drug database, the obtain alternative control parameter by analyzing may be increased to another infusion control parameter corresponding to the drug name. In this way, there are at least two infusion control parameters corresponding to the drug name in the drug database. For example, the drug name A in the drug database corresponds to an infusion control parameter 1 and an infusion control parameter 2, in which the infusion control parameter 1 may be a preferred infusion control parameter corresponding to the drug name A, and the infusion control parameter 2 may be a secondarily preferred infusion control parameter corresponding to the drug name A.

Of course, if the alternative control parameter corresponding to the drug name is associated with one or more of the patient type, the disease type, the medical history, the treatment history, the department information, the consumable type, the consumable model, and the consumable specification, then different infusion control parameters of a drug name may be set corresponding to a combination of one or more of the patient type, the disease type, the medical history, the treatment history, the department information, the consumable type, the consumable model, and the consumable specification.

It is to be noted that the aforementioned S101-S103 may be, but are not limited to, accomplished in the controller 201 of the infusion pump.

In summary, the embodiments of the invention provide a method for updating the infusion control parameters, so that the infusion pump can automatically obtain infusion records and/or medical advice information satisfying at least one of the selected analysis rules. At least one alternative control parameter corresponding to a drug name by analyzing the obtained infusion records and/or medical advice information, and the stored drug database of the infusion pump is updated according to the obtained alternative control parameter corresponding to the at least one drug name. There is no need for operation and maintenance (O&M) staff to collect drug information and infusion control parameters for infusion from doctors, pharmacists, nurses and other medical staff, thereby saving a lot of labor cost and time cost.

Further, on the basis of the embodiment corresponding to FIG. 1, after S102, the method provided by the embodiment of the invention further includes: outputting the obtained at least one drug name and its corresponding alternative control parameters.

For example, the obtained at least one drug name and its corresponding alternative control parameter may be output to one or more of the input-output device, the other infusion pumps, the workstation, and the central station, etc., so as to cause the other infusion pumps, the workstation, and the central station, respectively, to update the contained drug database according to the obtained at least one drug name and its corresponding infusion control parameter.

For example, on the basis of the embodiment corresponding to FIG. 1, the method provided by the embodiment of the invention further includes: outputting an alarm message when detecting that the frequency of use of the alternative control parameter corresponding to the at least one drug name in the drug database is lower than a set frequency threshold.

It is to be understood that when the frequency of use of the alternative control parameter corresponding to the at least one drug name is lower than the set frequency threshold, it indicates that the infusion control parameter corresponding to the drug name is not a commonly used infusion control parameter, and the medical personnel may have misconfigured the infusion control parameter of the drug name. In this circumstance, an alarm message of an abnormal setting of the infusion control parameter may be output by the input-output device 202 of the infusion pump to indicate for the medical personnel that there may be an error in the currently inputted infusion control parameter.

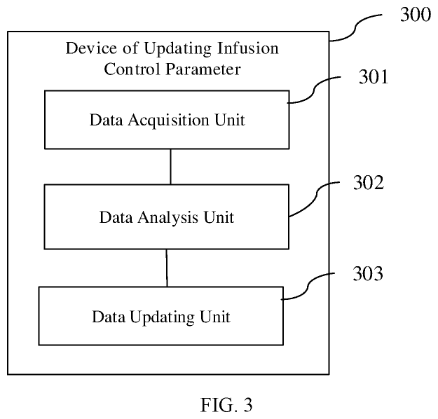

As shown in FIG. 3, the embodiments of the invention further provide a device 300 for updating an infusion control parameter, and it is to be noted that the basic principles and producing technical effects of the device 300 provided by the embodiments of the invention are the same as those of the embodiments described above. For the purpose of a brief description, the portions of the embodiments of the invention have not been referred to, may refer to the corresponding contents in the embodiments described above. The device 300 provided in the embodiments of the invention includes a data acquisition unit 301, a data analysis unit 302, and a data updating unit 303.

The data acquisition unit 301 is configured to obtain an infusion record and/or medical advice information in an infusion pump satisfying at least one selected analysis rule. The infusion record includes at least one drug name and a corresponding infusion record. The infusion record further includes at least one infusion control parameter, and the medical advice information comprises at least one drug name and a corresponding infusion control parameter.

The data analysis unit 302 is configured to analyze the obtained infusion record and/or the medical advice information to obtain the alternative control parameter corresponding to the at least one drug name.

The data updating unit 303 is configured to update the drug database stored in the infusion pump according to the obtained alternative control parameters corresponding to the at least one drug name.

In a possible embodiment, the data analysis unit 302 is configured to analyze the obtained infusion record and/or medical advice information to obtain the alternative control parameter corresponding to at least one drug name by: analyzing a change, and/or frequency of use of the at least one infusion control parameter corresponding to at least one the drug name in the obtained infusion record and/or medical advice information, to obtain the alternative control parameter corresponding to the at least one drug name; or, analyzing a difference between the at least one infusion control parameter in the infusion record corresponding to at least one drug name, and the infusion control parameter corresponding to the drug name in the medical advice information and/or the infusion control parameter corresponding to the drug name stored in the infusion pump to obtain the alternative control parameter corresponding to the at least one drug name; or, analyzing a difference between the infusion control parameter in the medical advice information corresponding to the at least one drug name and the infusion control parameter corresponding to the drug name stored in the infusion pump to obtain the alternative control parameter corresponding to the at least one drug name. The specific realization method thereof is as described above and will not be repeated.

In one possible embodiment, the infusion record includes patient information and/or infusion consumables information. The data analysis unit 302 is configured to analyze the obtained infusion records and/or medical advice information, and to obtain at least one alternative control parameter corresponding to the drug name, which is associated with one or more of patient type, disease type, medical history, treatment history, department information, consumable model, consumable type, and consumable specification. The specific implementation is described above and will not be repeated herein.

In one possible embodiment, the infusion record includes patient information, and the data analysis unit 302 is configured to analyze the obtained infusion record and/or medical advice information to obtain infusion control parameters corresponding to both patient information and the at least one drug name. The specific implementation method is described above, which will not be repeated herein.

In one possible embodiment, the infusion record may also include one or more items of infusion consumable information, including consumable type, consumable specification, consumable model, etc. The data analysis unit 302 is used to analyze the obtained infusion record and/or medical advice information to obtain infusion consumable information and infusion control parameters corresponding to at least one drug name. The specific implementation method is described above and will not be repeated herein.

In one possible embodiment, the selected analysis rules include one or more of an infusion time interval, an infused drug name, department information, a patient type, a disease type, a number of infusions, a frequency of changes to the infusion control parameter, a consumable type, a consumable model, a consumable specification, and a user trigger. The specific implementation method is described above, which will not be repeated herein.

In one possible embodiment, the data output unit is further configured to output an alarm message when detecting that the frequency of use of the alternative control parameter corresponding to the at least one drug name in the drug database is lower than a set frequency threshold

In one possible embodiment, the device 300 provided in the present embodiment further includes a data output unit configured to output the obtained at least one drug name and its corresponding alternative control parameters to one or more of the input-output devices, other infusion pumps, workstations, and central stations.

The present embodiment further provides an infusion pump, including a driving device, a casing, and a storage device provided within the casing, and a controller a controller configured to control the driving device to push out a liquid in an infusion consumable connected to the infusion pump, wherein the controller performs the method in the above-mentioned embodiments.

The present embodiment further provides a computer-readable storage medium, which stores a computer program. When the computer program is executed by the controller, the computer executes the method provided in the above embodiment of the invention.

The present embodiment further provides a computer program product, including a computer program that, when run, causes the computer to execute the method provided in the above embodiment.

Ordinary technical personnel in this field can understand that all or part of the steps to implement the above method embodiments can be completed through hardware related to program instructions. The aforementioned program can be stored in a computer-readable storage medium. When the program is executed, execute the steps including the above method embodiments. The aforementioned storage media include various media that can store program code, such as ROM, RAM, disks, or CDs.

Finally, it should be noted that the above embodiments are only used to illustrate the technical solution of the invention and not to limit it. Although detailed explanations have been provided for the invention with reference to the aforementioned embodiments, those skilled in the art should understand that they can still modify the technical solutions recorded in the aforementioned embodiments, or equivalently replace some or all of their technical features; And these modifications or replacements do not separate the essence of the corresponding technical solutions from the invention.

1. A method for updating an infusion control parameter comprising:
obtaining at least one of an infusion record collection and medical advice information in an infusion pump satisfying at least one selected analysis rule, wherein the infusion record collection comprises at least one drug name and a corresponding infusion record; the infusion record further comprises at least one infusion control parameter, and the medical advice information comprises at least one drug name and a corresponding infusion control parameter;
determining an alternative control parameter corresponding to at least one drug name by analyzing the at least one of the obtained infusion record and the medical advice information;
updating a drug database stored in the infusion pump according to the obtained alternative control parameters corresponding to the at least one drug name; and
selectively controlling the infusion pump according to the infusion control parameter and the alternative control parameter.

2. The method of claim 1, wherein the step of analyzing the at least one of the obtained infusion record and the medical advice information to obtain the alternative control parameter corresponding to at least one drug name comprises:
determining the alternative control parameter corresponding to the at least one drug name by analyzing at least one of a change in and a frequency of use of the at least one infusion control parameter corresponding to at least one the drug name in the at least one of the obtained infusion record and the medical advice information.

3. The method of claim 1, wherein the step of analyzing the at least one of the obtained infusion record and the medical advice information to obtain the alternative control parameter corresponding to at least one drug name comprises:
determining the alternative control parameter corresponding to the at least one drug name by analyzing a difference between the at least one infusion control parameter in the infusion record corresponding to at least one drug name, and the infusion control parameter corresponding to the drug name in the at least one of the medical advice information and the infusion control parameter corresponding to the drug name stored in the infusion pump.

4. The method of claim 1, wherein the step of analyzing the at least one of the obtained infusion record and the medical advice information to obtain the alternative control parameter corresponding to at least one drug name comprises:
determining the alternative control parameter corresponding to the at least one drug name by analyzing a difference between the infusion control parameter in the medical advice information corresponding to the at least one drug name and the infusion control parameter corresponding to the drug name stored in the infusion pump.

5. The method of claim 1, wherein the infusion record comprises at least one of patient information and infusion consumable information; the alternative control parameter is associated with one or more of a patient type, a disease type, a medical history, a treatment history, a departmental information, a consumable type, a consumable model, and a consumable specification.

6. The method of claim 1, wherein the selected analysis rule comprises at least one of an infusion time interval, an infused drug name, department information, a patient type, a disease type, a number of infusions, a frequency of changes to the infusion control parameter, a consumable type, a consumable model, a consumable specification, and a user trigger.

7. The method of claim 1, wherein after the step of analyzing the at least one of the obtained infusion records and medical advice information to obtain the alternative control parameters corresponding to the at least one drug name, and further comprising:
outputting the obtained at least one drug name and its corresponding infusion control parameter.

8. The method of claim 1, further comprising:
outputting an alarm message when detecting that the frequency of use of the alternative control parameter corresponding to the at least one drug name in the drug database is lower than a set frequency threshold.

9. The method of claim 1, further comprising:
outputting the obtained at least one drug name and its corresponding alternative control parameter to one or more of an input-output device, other infusion pumps, a workstation, and a central station.

10. An infusion pump comprising a driving device, a casing, a storage device arranged in the casing, and a controller configured to control the driving device to push out a liquid in an infusion consumable connected to the infusion pump, wherein the controller is configured:
to determine at least one of an infusion record collection and medical advice information in an infusion pump satisfying at least one selected analysis rule, wherein the infusion record collection comprises at least one drug name and a corresponding infusion record; the infusion record further comprises at least one infusion control parameter, and the medical advice information comprises at least one drug name and a corresponding infusion control parameter;
to determine an alternative control parameter corresponding to at least one drug name by analyzing the at least one obtained infusion record and medical advice information;
to update a drug database stored in the infusion pump according to the obtained alternative control parameters corresponding to the at least one drug name;
and to selectively control operation of the infusion pump according to the infusion control parameter and the alternative control parameter.

11. The device of claim 10, wherein the controller is configured to analyze the at least one obtained infusion record and medical advice information to obtain the alternative control parameter corresponding to at least one drug name by:
analyzing at least one of a change of and frequency of use of the at least one infusion control parameter corresponding to at least one the drug name in the at least one of the obtained infusion record and medical advice information, to obtain the alternative control parameter corresponding to the at least one drug name;
or, analyzing a difference between the at least one infusion control parameter in the infusion record corresponding to at least one drug name, and the infusion control parameter corresponding to the drug name in the at least one medical advice information and the infusion control parameter corresponding to the drug name stored in the infusion pump to obtain the alternative control parameter corresponding to the at least one drug name;
or, analyzing a difference between the infusion control parameter in the medical advice information corresponding to the at least one drug name and the infusion control parameter corresponding to the drug name stored in the infusion pump to obtain the alternative control parameter corresponding to the at least one drug name.

12. The device of claim 10, wherein the controller is configured to analyze the at least one obtained infusion record and medical advice information to obtain the alternative control parameter corresponding to at least one drug name by analyzing a difference between the at least one infusion control parameter in the infusion record corresponding to at least one drug name, and the infusion control parameter corresponding to the drug name in the at least one medical advice information and the infusion control parameter corresponding to the drug name stored in the infusion pump to obtain the alternative control parameter corresponding to the at least one drug name.

13. The device of claim 10, wherein the controller is configured to analyze the at least one obtained infusion record and medical advice information to obtain the alternative control parameter corresponding to at least one drug name by analyzing a difference between the infusion control parameter in the medical advice information corresponding to the at least one drug name and the infusion control parameter corresponding to the drug name stored in the infusion pump to obtain the alternative control parameter corresponding to the at least one drug name.

14. The device of claim 10, wherein the infusion record comprises at least one of patient information and infusion consumable information; the alternative control parameter is associated with at least one patient type, a disease type, a medical history, a treatment history, a departmental information, a consumable type, a consumable model, and a consumable specification.

15. The device of claim 10, wherein the selected analysis rule comprises at least one of an infusion time interval, an infused drug name, department information, a patient type, a disease type, a number of infusions, a frequency of changes to the infusion control parameter, a consumable type, a consumable model, a consumable specification, and a user trigger.

16. The device of claim 10, wherein the controller is further configured to analyze the at least one of the obtained infusion records and the medical advice information to obtain the alternative control parameters corresponding to the at least one drug name and to output the obtained at least one drug name and its corresponding infusion control parameter.

17. The device of claim 10, wherein the controller is further configured to output an alarm message when detecting that the frequency of use of the alternative control parameter corresponding to the at least one drug name in the drug database is lower than a set frequency threshold.

18. The device of claim 10, wherein the controller is further configured to output the obtained at least one drug name and its corresponding alternative control parameter to one or more of an input-output device, other infusion pumps, a workstation, and a central station, after analyzing the at least one of the obtained the infusion record and the medical advice information to obtain the alternative control parameter corresponding to the at least one drug name.
